(19) United States
(12) Patent Application Publication
(10) Pub. No.: US 2026/0102725 A1
(43) Pub. Date: Apr. 16, 2026

(54) Device for Medical Room Disinfection

(71) Applicant: Ecoviox International LLC, Miami, FL (US)

(72) Inventor: Juan Pablo Siri, Miami Beach, FL (US)

(21) Appl. No.: 19/104,991

(22) PCT Filed: Aug. 18, 2023
(86) PCT No.: PCT/US23/30587

§ 371 Date: Feb. 19, 2025

Related U.S. Application Data
(60) Provisional application No. 63/371,892, filed on Aug. 19, 2022.

(51) Int. Cl. B01D 46/00; A61L 2/104; A61L 2/202; A61L 2/24; A61L 9/015; A61L 9/20; A61L 9/22; A61L 101/02; A61L 103/40; A61L 103/75; B01D 53/04

(52) U.S. Cl. CPC B01D 46/0028; A61L 2/104; A61L 2/202; A61L 2/24; A61L 9/015; A61L 9/20; A61L 9/22; B01D 46/0002; B01D 46/0036; B01D 46/0047; B01D 53/0446; A61L 2101/02; A61L 2103/40; A61L 2103/75; A61L 2202/11; A61L 2202/14; A61L 2209/11; A61L 2209/12; A61L 2209/14; B01D 2253/102; B01D 2253/3425; B01D 2259/4508; B01D 2273/30; B01D 2279/35; B01D 2279/65

(57) ABSTRACT

A device for medical room disinfection that reduces or eliminates hospital-acquired infections that comprises: an external housing; an upper intake of polluted air; a lower outlet of purified air; a blower; at least one filter; at least one UVC lamp; an ozoniser; at least an ionizer.

### CROSS REFERENCE TO RELATED APPLICATIONS

This application is the United States national phase of International Application No. PCT/US23/30587, filed Aug. 18, 2023, and claims priority to U.S. Provisional Ser. No. 63/377,842 , filed Aug. 19, 2022, the disclosures of which are hereby incorporated by reference in their entireties.

### FIELD OF THE INVENTION

The present invention relates to devices and methods for purification, cleaning, disinfection and sterilization of air and surfaces in indoor environments. In particular, the present invention relates to devices and methods that are used in hospital rooms, particularly in intensive therapy (ICU) to eliminate or drastically reduce intra-hospital or hospital-acquired infections.

### STATE OF THE ART

It is known that numerous viruses, bacteria, fungi, spores, volatile organic compounds and particulate materials present in the air can cause bad odor and/or various health disorders such as bacterial infections, viral infections, allergies and other diseases. The air quality of the rooms where human beings perform their activities is of vital importance to maintain the safety and hygiene of such places.

On the other hand, the appearance of the new SARS-COV-2 coronavirus, which mainly spreads by airborne transmission by aerosols in the air, has renewed interest in the development of air and surface disinfection devices.

In this sense, numerous technologies, methods and strategies have been developed to eliminate air pollutants and disinfect rooms, however, all of them present some disadvantages.

Among the physical methods the use of HEPA filters can be mentioned. These filters consist of a physical barrier that is capable of stopping and retaining particles by different mechanisms. Other kinds of filters, such as activated carbon filters, are capable of absorbing certain molecules such as volatile organic compounds on their surfaces. Another widely used physical method is based on the use of ionizing radiation (such as UV light) for the elimination of microorganisms, which acts by removing the binding electrons of macromolecules that are irreplaceable for the life of cells, becoming unviable if the irradiated dose is high enough. In addition, through the use of air ionizers, it is possible to neutralize the electrostatic charges that keep the fine particles in suspension and therefore achieve their elimination.

On the other hand, among the chemical methods, the use of ozone is among the most relevant ones. This chemical compound is a strong oxidant. Its main advantage lies in the conversion of ozone into oxygen, without leaving any harmful chemical residue. More recently, the use of photocatalytic systems has been widely spread. UV light is used to promote the formation of highly reactive and short-lived chemical species on their surface, which can eliminate contaminants.

However, the use of these systems separately is usually not effective to achieve the goal of complete removal pollutants from the environment.

Among the main disadvantages of these systems are the low odor absorption capacity of HEPA filters and the possible proliferation of the microorganisms retained in the filters under the right conditions. UV lamps installed in air conditioning equipment usually do not achieve an adequate level of disinfection due to the high speed of the air flow that passes through the purification devices. Ozone, despite being an excellent disinfectant, is harmful in high concentrations and can irritate the upper respiratory tract in concentrations greater than 0.1 ppm. The patent application: US2022194180A1 DUAL DEVICE FOR ENVIRONMENT DISINFECTION AND STERILIZATION OF AIR AND SURFACES, from the same inventor of the present invention, introduces a device that combines some of these mechanisms in a way that when combined, allows for the treatment of air in commercial spaces, homes, and various facilities, improving air quality and facilitating the reduction of contaminant loads. Additionally, it presents a purification procedure using said device.

The issue in hospitals related to the number of deaths from nosocomial (hospital-acquired) infections is a challenging one to address. Current statistics from WHO indicate that more than 30% of patients admitted to intensive care units (depending on the quality of the hospitals) acquire nosocomial infections. This often becomes the leading cause of mortality under such conditions, with the death rate reaching 60% among those infected. This is a grave problem that has not found a solution in the current state of the art, despite all the investments made to control or mitigate it.

In the state of the art, some documents that describe methods and devices for disinfection of air wherein various technologies are combined can be found. For example, the document CN105737262 discloses an air purifier comprising an in-line intelligent induction and control device and guiding device, activated carbon filters, HEPA filters, an activated carbon photocatalyst, negative ion generator and ozone generator. In addition, the device autonomously detects the indoor air quality through various sensors and automatically adjusts the purification parameters of the equipment according to the air quality in automatic mode. However, it has an important disadvantage, being designed to be located on the floor of a room, its IR sensor may not detect the presence of living beings in their sterilization mode and seriously affect their health. On the other hand, it presents a major design problem; since ozone is denser than air, it has a short half-life and the device takes the air from a zone near the floor and drives it to a low height, ozone cannot reach greater heights and only what is below the outlet of the device is sterilized.

Document WO2018/106809, discloses a device that measures air quality parameters at the inlet and the outlet of the device and, based on the data collected, executes actions. In the detailed description of the invention, it is disclosed that said device may comprise technical characteristics similar to those previously mentioned. However, the document does not disclose how to order the various cleaning and disinfection methods mentioned to maximize their efficiency.

The utility model CN203785115 U provides a negative ion air purifier, said device comprises a fan, a negative ion generator, HEPA filters, activated carbon fiber filters, a multiplicity of UV lamps, and an antibacterial filter with silver nanoparticles.

In addition, state-of-the-art devices that purify the air from the ground present the impossibility of aspiring the contaminated air in height and returning it purified to the level of the respiratory tract. During the day, human beings generate aerosols at greater heights than 1.6 meters, and they breathe the contaminated air at the same height.

The state of the art shows technological attempts to purify the air in hospital rooms and intensive care rooms, such as CN111998469 and CN216667924. These patent documents teaches combinations of known operations that do not completely reduce nosocomial infections.

As can be seen, there is still a need for an air and surface purification device and method, installable in indoor environments, that combines different decontamination strategies in such a way as to ensure maximum disinfection and air quality. In addition, the problem of intra-hospital contamination has not been solved so far in the world.

The present invention solves the problems identified in the state of the art, providing a device that comprises an external housing with mirrored inner surfaces; a upper intake of polluted air; a lower outlet of purified air; a centrifugal blower with two air vents; at least one HEPA filter; at least one activated carbon filter; at least one UV lamp; an ionizer positive, and preferably one negative ion generator; preferably located higher than 2 m. This device drastically lowers the contamination, eliminating organic compounds, microorganisms, viruses and bacteria.

The present invention provides a device for disinfection of medical rooms that allows to reduce and even eliminate hospital-acquired infections.

The present invention provides a device for disinfection of medical rooms that turns on the ozone generator between 2 and 20 minutes for every 4 hours of operation, managing to eliminate contaminants and stay below the maximum ozone concentrations allowed in work environments.

The present invention provides a disinfection process for hospital rooms, particularly intensive care units, which is carried out using the device of the present invention and operates continuously for all its components, except for the ozone generator, which operates only between 2 and 20 minutes every four hours.

In one embodiment, the device of the invention comprises two ionizers, one that generates only negative ions and is located in the suction of one of its centrifugal blowers, and another that generates positive ions and is situated in the suction of the second centrifugal blower. Both air streams exit separately through two vents on the lower output of the invention device. This creates two parallel flows that enter the room and disinfect with such efficiency in their disinfecting capacity that the use of photocatalytic filters is not necessary.

None of the documents from the prior art provide a device that achieves the results that the present invention has demonstrated. The combination of its passive components (filters) and active ones (UV lamp, ozone generator, ionizers), generating positive ions through one output vent and producing negative ions through the other output vent, achieve extraordinary efficiency in protecting the health of both patients and intervening staff.

No air purifier for medical rooms in the state of the art proposes the intermittent use of ozone in such a way that the internationally accepted maximum ozone levels for a workday are not exceeded.

Scientific study carried out by the most outstanding medical and scientific professionals of the University of Buenos Aires and the Hospital de Clínicas Argentina. The scientific study was carried out in highly complex environments in order to determine the degree of efficiency that the device for medical room disinfection known as Ecoviox Medical Grade air and surface sterilizers have in reducing hospital-acquired infections. The study has 12 months of work, three reports were issued, that complete the year of study. After one year of operation in 18 hospital areas, the result was a 100% efficiency reduction in hospital-acquired infections. Prior to the installation of the devices of the present invention the annual average of hospital-acquired infections was 40%. Taking into account that about 180 patients passed through the areas of Immosuppressed patients in a year, about 80 patients were prevented from becoming infected, allowing them to recover from their pathologies, avoiding morbidity and mortality, reducing the days of hospitalization, drastically reducing the hospital costs. As a conclusion, the device for medical room disinfection known as medical grade Ecoviox technology was able to eradicate nosocomial infections with 100% effectiveness.

### BRIEF DESCRIPTION OF THE FIGURES

FIG. 1: Front view of the device of the invention, in a preferred embodiment, where you can observe an external housing(1); a top inlet for contaminated air (2); a bottom outlet for purified air (3); a blower (4); the activated carbon filter (5), the HEPA filter (6); the UVC lamp (7); the ozonizer (8) which is a UVC O3 lamp; the ionizers, the positive (9) and the negative (10); The blower motor (11), the two centrifugal turbines (12), and the two bottom windows for the outlet of purified and/or sterilized air.

FIG. 2: Side view where can be seen the same elements as in FIG. 1.

FIG. 3: The air flows in the device of the present invention are observed: the entry through the top, the exit at the bottom of two parallel purified air streams, one with exclusively negative ions and the other with positive ions. Additionally, you can see how after passing through the UVC lamps the entered stream splits in two.

FIG. 4: Normal operation when the UVC lamp is turned on.

FIG. 5: Operational situation that occurs every 4 hours for 10 minutes where the ozonizer is turned on.

FIG. 6: Shows the suggested configuration to place the device of the present invention in an intensive care room.

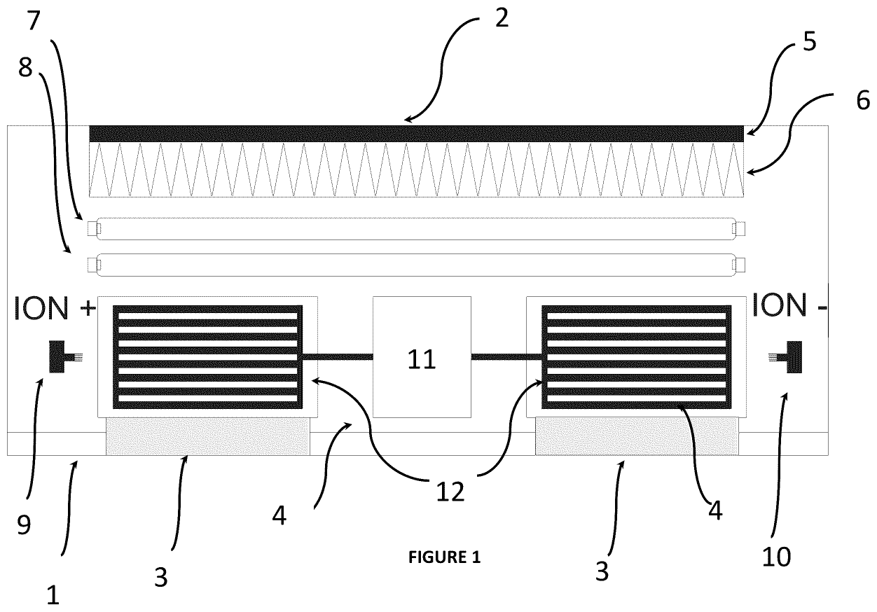

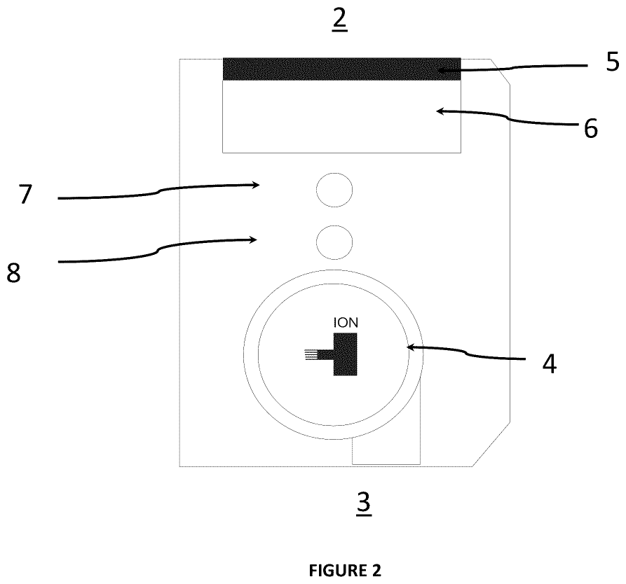

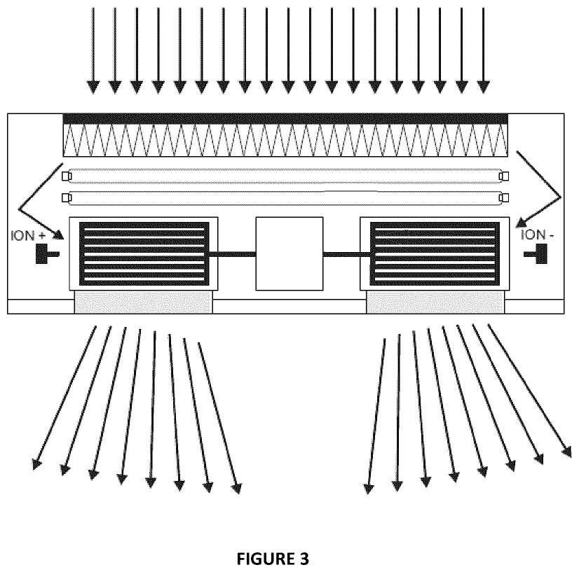

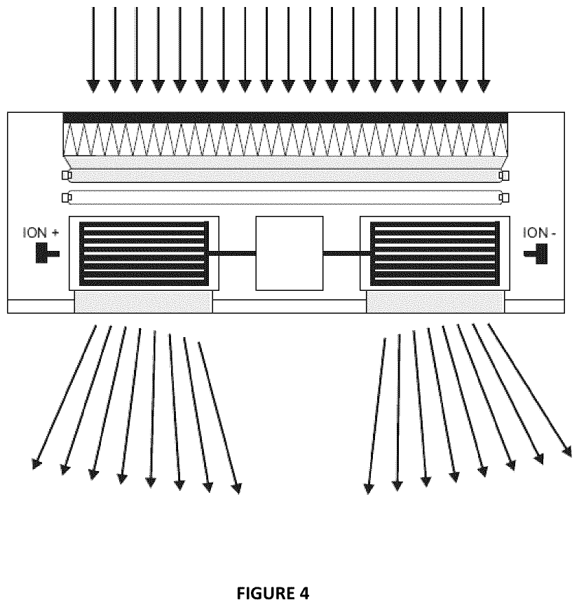

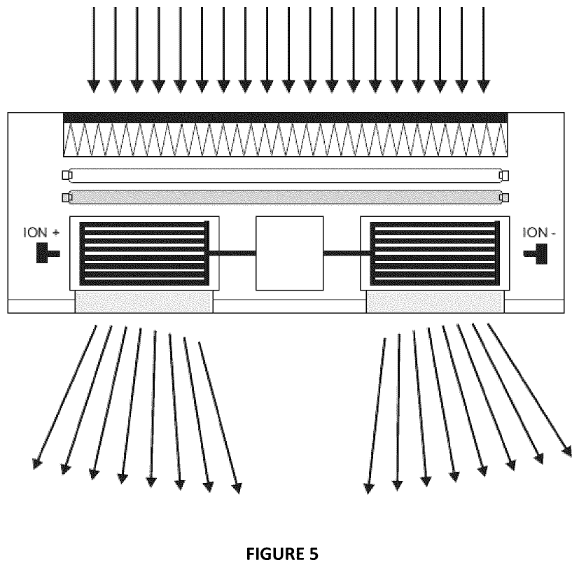

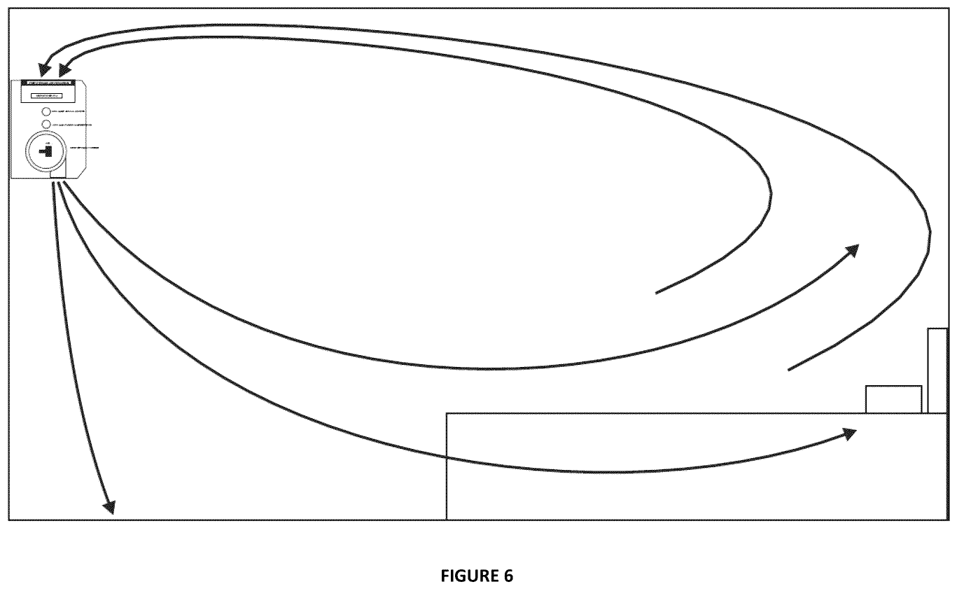

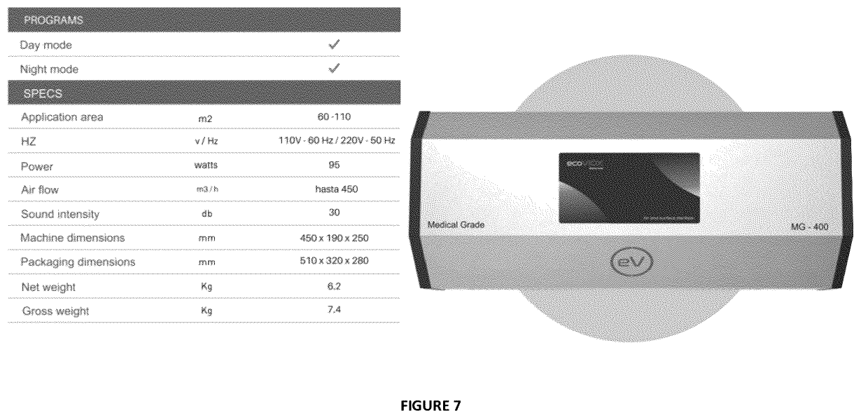

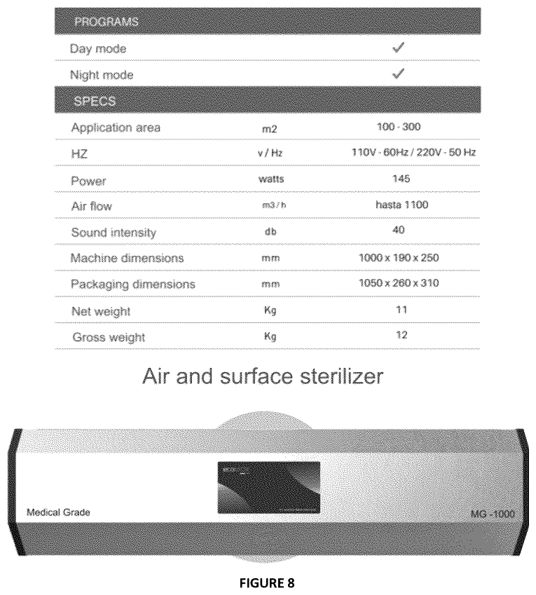

### BRIEF DESCRIPTION OF THE INVENTION

The medical room disinfection device of the present invention comprises: an external casing; a top inlet for contaminated air; a bottom outlet for purified air; a blower; at least one filter; at least one UV lamp; an ozonizer; and at least one positive ionizer. Wherein, in one embodiment, said filter comprises Honeycomb-type Activated Carbon Filters and a HEPA filter with a minimum width of 5 cm and a double silk pre-filter; said ozonizer comprises a UVC O3 Lamp; where said lamp comprises one germicidal UVC lamp of 254 nm of at least 80 W.

In one embodiment, the present invention also comprises a negative ionizer, generating only negative ions.

Wherein the top inlet for contaminated air is located in the top section of said housing and the bottom outlet for purified air is located in the bottom section of said casing, where the bottom outlet comprises two exit windows for air driven by two centrifugal turbines that suction, one with exclusively negative ions and the other with positive ions. Where said positive ionizer generates negative ions and positive ions in a ratio from 1 to 3/2 as negative ions/positive ions. And these ionizers are of plasma type.

The invention's device operates at a height of at least two meters, drawing air from its top and directing the purified air downwards.

In a preferred embodiment of the invention, said activated carbon filter comprises granular, fibrous, or powdered activated carbon; and the grades of the HEPA filters are selected from the group consisting of H11, H13, and H14. Wherein these UV lamps are selected from the group consisting of: LED technology lamps and mercury vapor lamps.

In a preferred embodiment of the invention, the medical room disinfection device comprises: an external housing; a top inlet for contaminated air; a bottom outlet for purified air with two windows; an electronic controller with a timer, a blower with two centrifugal turbines; a HEPA filter and an activated carbon filter; at least one UV lamp; an ozonizer; a positive ionizer located in the suction of one of the centrifugal turbines and a negative ionizer located in the suction of the other centrifugal turbine. Wherein said electronic controller with a timer controls the intermittent operation of the ozonizer so as not to exceed the maximum ozone concentration allowed in a working day.

In a preferred embodiment of the present invention, said housing with mirrored internal surfaces comprises polished metal at least on its internal surface or polished stainless steel at least on its internal surface.

In a preferred embodiment of the present invention, said blower comprises two centrifugal turbines commanded by a single electric motor located between said turbines that operates at about 2800 rpm and generates an airflow of from 400 to 2000 m3/h.

In a preferred embodiment, the present invention comprises a positive ionizer and another negative ionizer that are placed in the suction of each centrifugal turbine, maintaining two air streams with different charges.

Another object of the present invention is a method for air disinfection in medical rooms using the device of the invention, wherein said device is preferably installed at a distance of at least 2 m and less than 5 m from the ground and comprises the following steps:
a. drawing the air from the medical room to be purified through the top inlet;
b. forcing the air drawn in step “a” to pass through the Honeycomb-type activated carbon filter, then through the pleated HEPA h14 filter of at least 5 cm thick, through a UV lamp of at least 80 W;
c. splitting the air stream in two, where one of the two streams passes through a positive ionizer and the other stream passes through a negative ionizer;
d. turning on the ozonizer no more than 20 minutes every 4 hours;
e. expelling the purified, ionized, and disinfected air through two bottom exit windows, generating two parallel streams, one carrying positive ions and the other carrying only negative ions.

Wherein said method, said UV lamp is UVC and irradiates said HEPA filter. And wherein the suction of step “a” is carried out by a blower comprising a central motor that moves two centrifugal turbines that split the airflow in two. Wherein these two parallel streams comprise a stream with only negative ions and the other stream with both positive and negative ions.

### DETAILED DESCRIPTION OF THE INVENTION

The medical room disinfection device of the present invention combines both its passive components (filters) and active components (UV lamp, ozonizer, ionizer), integrating the generation of positive ions through one outlet vent, and in one embodiment, the generation of negative ions through the other outlet vent. This achieves remarkable efficiency in safeguarding the health of both patients and attending staff.

In this document, the term “medical room” refers to all enclosed spaces where actions related to health restoration are carried out, including but not limited to waiting rooms, operating rooms, consultation rooms, intensive care units, first aid rooms, primary care rooms, emergency rooms, etc.

Preferably, the device is comprised of a blower or mechanism to move the air, a combination of activated carbon filters, HEPA filters, UV lamp, ionizer, and ozonizer.

Additionally, in one embodiment, the device of the present invention includes an electronic controller with a timer, a touchscreen that allows for the operation of the equipment to be controlled and monitored, tracking its operation, temperature, program, and air condition; a remote control, for conducting all the basic operations remotely; and brackets for its installation on a wall.

The device of the present invention also encompasses an external housing that contains all the contaminant retention and removal systems, the mechanism for moving the air, and all the necessary electronics for its operation. This housing includes mirrored interior surfaces and is designed in such a way that it can reflect the ultraviolet light emitted by the UV lamps, maximizing their efficiency while preventing direct radiation exposure to humans.

The device works in such a way that ambient air is aspired into the housing through an upper intake of contaminated air located at upper section of the device, thanks to the pressure gradient generated by the mechanism to displace air (blower). Then, the air passes through the activated carbon filter, and the HEPA filter. Once inside the device, the air is irradiated with ultraviolet light. In turn, the ultraviolet lamp irradiates the HEPA filter to prevent the proliferation of fungi and bacteria in it. Inside the housing, positive ions are introduced into the air by the ionizer and ozone is introduced by the action of the ozone generator. Finally, the filtered, disinfected and loaded with ions and ozone air, interacts with the blower, which forces it out of the housing through a lower outlet of purified air located at the lower section of the device.

In this document, disinfection is understood as any action executed to reduce the load of viruses, bacteria, fungi, microorganisms, organic gases and particles. It is understood from a general perspective, also including the concept of purification and sterilization.

### Activated Carbon Filters

Activated carbon is a highly porous material that, through the phenomenon of surface adsorption, retains gases and vapors from the air, for example, volatile organic compounds, radon, and NO₂, among others.

In a preferred embodiment of the present invention, the activated carbon filters comprise granular activated carbon housed in a honeycomb-like cell structure, where the volume of said cells is partially filled (20% of the volume of the cell) with activated carbon granules. In this way, a low-pressure drop and adequate distribution of the granules is achieved as a result of a homogeneous thickness of adsorbent material. The formation of plugs or preferential channels for the passage of gases is also avoided.

In another embodiment, the activated carbon filter comprises the use of activated carbon fibers. This morphology has unique characteristics compared to granular or powdered activated carbon. The thin fibrous shape ensures faster absorption and is very easy to handle.

### Hepa Filters

HEPA (High-Efficiency Particulate Arresting) filters are composed of a mat of randomly arranged fibers. The fibers are typically composed of fiberglass, cotton or polyester, with diameters between 0.5 and 2.0 μm. HEPA filters are designed to effectively stop very fine particles, but they do not retain gases or odor molecules.

In the device of the present invention, the HEPA filters are irradiated by an ultraviolet lamp that produces light in a wavelength range of 254-280 nm. This germicidal process generates the total inactivation of the viruses and bacteria that are trapped in the fibers of the HEPA filter. If this inactivation is not generated, mold, yeasts and bacteria generate colonies in the filters and over time they can return to the environment. The device of the present invention comprises HEPA filters wherein said HEPA filters grades are selected from the group comprised by H11, H13 and H14. In a preferred embodiment, it uses HEPA H14 filters.

### Ultraviolet Light

Ionizing electromagnetic radiation in adequate doses can generate cellular damage by direct action (damage to important macromolecules such as DNA, RNA, enzymes) or mechanisms that involve indirect action (damage to the cell membrane, formation of free radicals by homolytic breakdown of the water present in the cytoplasm, etc.). When the radiation dose is high enough, the damage to the cells is too great and they are unable to repair themselves, becoming unviable. The UV lamps of the present invention are located in such a way that, in their position, the radiation received by the HEPA filter exceeds 50 W/cm² and in turn irradiates the turbine rotor of the mechanism to displace air eliminating any microorganism that adheres to it, thanks to the interior mirrored surface of the housing.

Said UV lamps comprise LED technology lamps or mercury vapor lamps. In one embodiment, said UV lamps comprise UVC-FAR type lamps. In a preferred embodiment, said UV lamps are of the UVA/UVC type with LED technology.

In this document, a positive ionizer is understood to mean those ionizers that generate positive ions as well as negative ones. Preferably the ratio between negative ions/positive ions generated is from 2/3 to 3/2.

While a negative ionizer is understood to be the one that only generates negative ions.

Dust particles, pollen, bacteria and other allergens have a positive surface charge and are capable of remaining in suspension in the environment thanks to electrostatic interactions. Negative ions act by neutralizing this charge and sometimes causing their agglomeration, promoting their precipitation due to gravity.

The ionizer comprises a plasma anion generator. In addition to eliminating 99% of all suspended particles of less than 2.5 microns, plasma can neutralize viruses and bacteria by destroying their molecular structure.

### Ozone

Ozone (O₃) is a well-known disinfecting agent which has been widely used in various applications because it is a highly oxidizing compound that destroys organic substances, bacteria molecules, molds, etc. ; therefore, it is capable of sterilizing the air and eliminating toxic gases and odors. Ozone owes its great oxidizing capacity to the fact that its third oxygen atom is a loose radical that reacts with volatile organic compounds, neutralizing odors and certain gases, producing oxygen (O₂) as the main by-product. It has germicidal properties and is capable of destroying all kinds of toxins, bacteria, fungi and viruses present in an environment. Being a gas, it is capable of facilitating disinfection tasks in upholstery and fabrics as well as in areas of difficult access.

In one embodiment of the invention, the ozone generator of the invention comprises corona discharge ozone generators. In another embodiment of the invention, the ozone generator comprises UVC-O3 lamps specifically designed to maximize ozone generation.

The density of ozone is 2.1 kg/m³ while that of air is only 1.2 kg/m³. For this reason, many of the devices that constitute the state of the art do not work, especially if they are located on the floor.

The device of the present invention is designed to operate at a height of more than two meters and has directing fins to direct the purified air that comes out from under said housing in various directions and at angles close to horizontal to achieve its optimal distribution. In this way the ozone falls on the surfaces of furniture and objects, sterilizing them.

### Blower

The device of the invention also has a mechanism to displace air, or blower, which comprises an electric motor that is attached to turbines, displacing the air necessary for the correct operation of the device of the present invention. In a preferred embodiment of the present invention, said mechanism to displace air comprises an electric motor with two turbines. In an even more preferred embodiment of the present invention, the device comprises a blower (similar to those used in air conditioning equipment) composed of two centrifugal turbines controlled by a single electric motor located between said turbines that operate at around 2800 rpm. According to this embodiment, the turbines comprise an inner housing and a multiplicity of blades.

In one embodiment, the device of the invention comprises at least a mode of operation: the UV lamps and the ionizer operate, while air is circulated through the activated carbon, HEPA filter. While the ozone generator operates only a few minutes (preferably from 2 to 20 min) each 4 hours. Another object of the present invention is a method of purification, cleaning and disinfection of air and surfaces for hospital rooms that uses the device of the present invention, characterized in that said device is preferably installed at a minimum distance of 2 m from the floor and comprises the following steps:
a. aspiring the air from the room to purify through a top intake;
b. forcing the air aspired in step “a” to pass through the activated carbon filter, through the HEPA filter, through a UV lamp, through the positive ionizer and
c. expelling the purified and disinfected air through a bottom outlet.
d. turn on the ozone generator no more than 20 minutes each 4 hs.

In a preferred embodiment of the present invention in step “b” in addition, after de UV lamp the air stream is split into two streams, where one of the two streams passes through a positive ionizer and the other stream passes through a negative ionizer; and each of these two air streams are propelled out of the device throw two independent exhaust vents, one with positive ions and the other with negative ions.

Furthermore, said method is characterized in that it is a method of sterilizing the air in the room where it is installed, which comprises the automatic operation of said ozone generator wherein the automatic control turns on said ozonizer no more than 20 minutes each 4 hours. Ensuring that the ozone concentration throughout the workday does not exceed the allowed maximum limits.

The device of the invention is preferably installed at a height of between 2 and 2.5 m from the ground, always preferably less than 5 m. In an even more preferred embodiment of the invention, the device of the invention is installed at a minimum distance of 2.3 m from the ground.

A method of implementing the present invention for a preferred embodiment thereof is described below:

HEPA H14 Filter Assembly: The HEPA H14 filter is a key component, as it can capture very fine particles, including viruses and bacteria. Prefilters and activated carbon are also added to enhance the filter's efficiency.

Centrifugal Turbine Installation: A high-flow centrifugal turbine is installed to ensure adequate airflow through the sterilizer. This turbine operates at low decibels, and in its preferred form, features an adjustable rotor speed to cater to varying needs.

UVC Lamp Installation: A UVC lamp is installed, which emits ultraviolet light that significantly reduces or eliminates microorganisms, especially viruses and bacteria in the air and on surfaces.

UVC O3 Lamp Installation: In addition to the UVC lamp, a UVC O3 lamp producing ozone is set up.

Positive Ion Generator Installation: A bipolar positive ion generator produces both positive and negative ions that attract and neutralize airborne contaminants.

Negative Ion Generator Installation: In addition to the positive or bipolar ion generator, a negative ion generator is installed to produce even more negative ions, enhancing air quality.

Installation of the Process Control Board with Timer: This is set up to control and monitor the device's operation. Specifically, this timer triggers the ozonizer to run for 2 to 20 minutes every 4 hours, preferably 4 minutes every 4 hours.

The assembly of the equipment begins by placing the lower part of the cabinet, housing the electromechanical components, on a workbench. The first step involves installing the centrifugal turbine, which has a central motor and two lateral centrifugal systems (turbines). Following this, the air ionizers are installed: the negative ionizer on the right nozzle of the turbine and the positive (bipolar) ionizer on the left, achieving two independent and parallel air flows-one with a 100% negative ion charge and the other with both positive and negative ions.

Subsequently, the UVC and UVC O3 lamps are installed. These lamps are fixed at the top of the cabinet onto flexible bases. Once the lamps are set up, the electronic ballasts, which serve as the lamps'starting system, are anchored and connected.

Above the lamps, filters-both HEPA and activated carbon—are mounted.

The invention's device has drastically reduced hospital-acquired infections in intensive therapy or intensive care units (ICU). as illustrated in the following examples.

### EXAMPLES

### Example 1

An Example of the Present Invention Is Described in Detail Below Without Intending to limit the claims.

Construction of device for medical room disinfection of the present invention.

The device of the present invention was built from a stainless-steel support, in which, a UV Led lamp, a UVC O3 lamp as ozone generator and two plasma ionizers were fixed by specially designed screws and sockets. Said support is attached to a main body by welding, allowing the device to be attached to a wall by using bolts. A stainless-steel housing polished on its inner surfaces closes the device and is attached by pressure fittings and security screws to said support. On the horizontal face of the housing, a CPU with a touch screen was attached to the outside of the device, which allows controlling the operation of the equipment and allows processing the information from the temperature and air condition sensors. In addition, this CPU is controlled remotely or by touch, to execute all the basic operations remotely.

Said housing contains all the contaminant retention and elimination systems, the mechanism to displace air and all the necessary electronics for its operation. Inside, it comprises mirrored stainless steel inner surfaces and is designed in such a way that it is capable of reflecting the ultraviolet light emitted by the lamps, maximizing their efficiency.

Said activated carbon filter comprises granular activated carbon housed in a honeycomb-like cell structure, where the volume of said cells is partially filled (20% of the volume of the cell) with activated carbon granules. This avoids the reduction of the air flow and inactivates harmful gases. In addition, said filter is located next to the upper intake of contaminated air of the housing.

The HEPA filter comprises H14 type filter panels of 6 cm with that and retain 99.995% of the particles and microorganisms from the air that passes through it.

In addition, said UV lamp is a UVC lamp of 254 nm of 80 W. This lamp is located in such a way that in its position the radiation received by the HEPA filter exceeds 50 W/cm². The mechanism to displace air is a motor that drives two centrifugal blowers and that rotates at 2800 rpm. Regarding the ionizers, one is a positive plasma ion generator, while the second one is a negative ion plasma generator.

The characteristics of these ionizers are as follows: Positive or bipolar ion generator: Input voltage: 12 V DC, negative ion concentration: −4 million; positive ion concentration: +6 million; negative voltage output: −4.0±0.5 KV; positive voltage output: +3.0±0.5 KV.

High density negative ion generator: operation 12 V DC, maximum load 3 W; high negative voltage output −19.5 KV; dimensions: 1.1 inches wide×2.2 inches long×1.2 inches high; negative ion density 6 million.

The device works in such a way that ambient air is aspired into the housing through an upper intake of contaminated air located at upper section of the device, thanks to the pressure gradient generated by the mechanism to displace air.

Regarding the mechanism to displace air, it comprises an electric motor that is bolted. This motor rotates two shafts driving separate centrifugal blowers. Said blowers are two centrifugal turbines controlled by a single electric motor which is located between said turbines. Said turbines comprise an inner housing and a multiplicity of blades. Said mechanism is located close to the air outlet of the housing.

The air flow that is achieved with these turbines could exceeds 2000 m³/h, but could be regulated.

Each ionizer is placed in the suction of each centrifugal blower. Each of said centrifugal blower forces clean air loaded with ions of different charge out of the housing through two separated bottom outlets of purified air located at the lower section of the device.

The device of the invention comprises an operation mode that, in combination with its technical characteristics, could eliminate hospital-acquired infections: The UV O3 lamp is on 4 minutes each 4 hours of operation. The UV lamp and ionizers operate continuously, and air is circulated through the activated carbon, HEPA filters.

### Example 2

An Example of the Present Invention Is Described in Detail Below Without Intending to limit the claims.

Construction of device for medical room disinfection of the present invention.

The device of the present invention was built from a stainless-steel support, in which, a UVC lamp, a UVC 03 lamp as ozone generator and two plasma ionizers were fixed by specially designed screws and sockets. Said support is attached to a main body by welding, allowing the device to be attached to a wall by using bolts. A stainless-steel housing polished on its inner surfaces closes the device and is attached by pressure fittings and security screws to said support. On the horizontal face of the housing, a CPU with a touch screen was attached to the outside of the device, which allows controlling the operation of the equipment and allows processing the information from the temperature and air condition sensors. In addition, this CPU is controlled remotely or by touch, to execute all the basic operations remotely.

Said housing contains all the contaminant retention and elimination systems, the mechanism to displace air and all the necessary electronics for its operation. Inside, it comprises mirrored stainless steel inner surfaces and is designed in such a way that it is capable of reflecting the ultraviolet light emitted by the lamps, maximizing their efficiency.

Said activated carbon filter comprises granular activated carbon housed in a honeycomb-like cell structure, where the volume of said cells is partially filled (20% of the volume of the cell) with activated carbon granules. This avoids the reduction of the air flow and inactivates harmful gases. In addition, said filter is located next to the upper intake of contaminated air of the housing.

The HEPA filter comprises H14 type filter panels of 6 cm with that are capable of retaining 99.995% of the particles and microorganisms from the air that passes through it.

In addition, said UV lamp is a UVC lamp of 254 nm of 80 W. This lamp is located in such a way that in its position the radiation received by the HEPA filter exceeds 50 W/cm². The mechanism to displace air is a motor that drives two centrifugal blowers and that rotates at 2800 rpm. Regarding the ionizers, one is a negative plasma ion generator, while the second one is a positive ion plasma generator.

The device works in such a way that ambient air is aspired into the housing through an upper intake of contaminated air located at upper section of the device, thanks to the pressure gradient generated by the mechanism to displace air.

Regarding the mechanism to displace air, it comprises an electric motor that is bolted. This motor rotates two shafts driving separate centrifugal blowers. Said blowers are two centrifugal turbines controlled by a single electric motor which is located between said turbines. Said turbines comprise an inner housing and a multiplicity of blades. Said mechanism is located close to the air outlet of the housing.

The air flow that is achieved with these turbines could exceeds 2000 m³/h.

Each ionizer is placed in the suction of each centrifugal blower. Each of said centrifugal blower forces clean air loaded with ions of different charge out of the housing through two separated bottom outlets of purified air located at the lower section of the device.

The device of the invention comprises an operation mode that, in combination with its technical characteristics, could eliminate hospital-acquired infections: The UV O3 lamp is on 10 minutes each 4 hours of operation. The UV lamp and ionizers operate continuously, and air is circulated through the activated carbon and HEPA filter.

### Example 3

Trial at the Hospital de Clínicas de la UBA in the city of Buenos Aires

Use of the device for the disinfection of medical rooms of the invention REPORT ROOM 3 OF IMMUNOSUPPRESSED (IS) PATIENTS.

PROF. Dra. SOFíA ISABEL FERNáNDEZ

EVALUATION OF THE DEVICE FOR MEDICAL ROOM DISINFECTION OF THE EXAMPLE 1 OF THE INVENTION.

### Population to Be Evaluated:

Neutropenic patients, with problems in the quantity or quality of their neutrophils, secondary to chemotherapy or underlying disease (leukemia).

### Introduction:

Patients are those who have altered some of their defense mechanisms, whether linked to their natural or adaptive immunity.

Neutropenia is the decrease in the function of the neutrophil (natural immunity) due to disorders in its quantity or quality. Regarding the quantity according to IDSA 2011, a decrease below 500/mm³ is required or in quality, which is the presence of blasts, in connection with leukemia.

Although gram-negative sepsis, especially due to pseudomonas, increases the morbidity and mortality of these patients, there are other infections that place them in a condition of high vulnerability and poor prognosis.

The historical experience in terms of our epidemiological data is the increase in pulmonary infection due to probable invasive aspergillosis (it is sufficient even without the certainty provided by lung biopsy), to speak of said infection, the image plus the curve of galactomannans in blood/BAL, and a history of profound and prolonged neutropenia, are adequate for such a diagnosis.

The arrival of the spores of said fungus to the lung of the patient with these conditions is an unavoidable situation in our environment due to the environmental characteristics, devoid until March of filters to sterilize the air of said germs.

Characteristically, the neutrophil of the immunocompetent host has an enzyme system that, upon coming into contact with said fungus, manages to phagocytose and eliminate it, a situation that cannot be implemented in the immunosuppressed patient due to neutrophil dysfunction, which lacks said enzyme system, in quantity (absolute neutropenia) or quality (presence of blasts), where an immature system makes such purification impossible.

Pulmonary aspergillosis increases the morbidity and mortality of patients with IS. In addition to being a serious pathology for this population, it slows down chemotherapy treatment, that is, it worsens the prognosis of the underlying disease, as well as causing superinfections and if the patient is requiring a transplantation, it is a factor that makes such a procedure difficult in the short term and long term.

As it was presented, in a normal situation this infection is increasing, as evidenced by our statistics.

Starting in March, three filters were placed in 3 rooms, visible for the admission of neutropenic patients.

From that month to June included, there were no evidence of pulmonary infections through upper airway access in the rooms with filters.

A single patient developed a pulmonary nodule probably originating from a skin and soft tissue infection (pathology with which he had been admitted).

THERE WAS NO EVIDENCE OF PULMONARY ASPERGILLOSIS IN THIS GROUP OF PATIENTS SINCE THE OPERATION OF THESE FILTERS, OR OTHER LUNG INFECTIONS.

In this same period, the presence of patients with pulmonary aspergillosis had been documented, among other IH infections (acquired within the hospital) in patients with SI (before the placement of the devices for the disinfection of medical rooms of the invention)

As shown, its presence is not only related to morbidity and mortality, but also to increased costs, greater hospitalization and, above all, the cost of the antifungals used, which together with the oxygen that is consumed to a great extent in this context, and the most expensive antibiotics.

It should be noted that the arrival of covid through the pandemic is also a risk factor for acquiring said fungus, considering that our patient population has a higher risk of acquiring the virus, even vaccinated.

The only way found to prevent the arrival of these spores, viruses and bacteria is by filtering them into the ambient air using the device for disinfection of medical rooms of the invention.

### Example 4

REPORT ROOM 3 OF IMMUNOSUPPRESSED PATIENTS (IS), FOURTH CHAIR OF MEDICAL CLINIC. PROF. DRA SOFÍA ISABEL FERNANDEZ EVALUATION OF THE DISINFECTION AND STERILIZATION SYSTEM OF AIR AND SURFACES, device of the present invention: ECOVIOX. Population to be evaluated: Neutropenic patients, with alteration in quality or quality of their neutrophils, secondary to chemotherapy or underlying disease (leukemia).

Introduction: IS patients are those who have altered some of their defense mechanisms, either linked to their natural or adaptive immunity. Neutropenia is the decrease in neutrophil function (natural immunity) by disorders in its quantity or quality. As for the quantity according to IDSA 2011, a decrease below 500/mm3 or in quality, which is the presence of blasts, is required in connection with leukemia. Although sepsis at gram negative, especially pseudomonas increases the morbidity and mortality of these patients, there are other infections that position them in a condition of high vulnerability and poor prognosis. The historical experience regarding our epidemiological data is the increase in lung infection by probable invasive aspergillosis (it is sufficient even without the certainty that lung biopsy gives it), to talk about this infection, the image plus the curve of galactomannans in blood/BAL, and the antecedent of deep and prolonged necropopenia, are suitable for such a diagnosis. The arrival of the spores of this fungus to the lung of the patient with these conditions, is an inevitable situation in our environment due to the environmental characteristics, devoid until March of filters to sterilize the air of these germs. Characteristically, the neutrophil of the immunocompetent host has an enzyme system Which, when in contact with this fungus, manages to phagocytize and eliminate it, a situation that cannot be implemented in the immunosuppressed patient due to neutrophil dysfunction, who lacks this enzyme system, in quality (absolute neutropenia) or quality (presence of blasts), where an immature system makes such debugging impossible. Pulmonary aspergillosis increases morbidity and mortality in patients with IS. In addition to being a serious pathology for this population, it slows down chemotherapy treatment, that is, it worsens the prognosis of the underlying disease, as well as leads to superinfections and requiring MO transplantation, it is a factor that hinders such a procedure in the short and long term. As mentioned, if we do not have adequate environmental filters, this infection is increasing, as evidenced by our statistics. As of March, three filters were placed in their respective rooms, assigned for the admission of neutropenic patients. From that month to June inclusive, lung infections through upper airway access were not evident in the rooms with filters. Only one patient developed a pulmonary nodule of probable arrival from skin and soft tissue infection (pathology with which he had been admitted).

THERE WAS SO FAR NO EVIDENCE OF PULMONARY ASPERGILLOSIS IN THIS GROUP OF PATIENTS FROM THE ACTION OF THESE FILTERS. OR OTHER LUNG INFECTIONS.

In this same period, the presence of patients with pulmonary aspergillosis, among other IH infections (acquired within the hospital) in patients with IS (before their placement) had been documented. As mentioned, not only does its presence have to do with morbidity and mortality, but also the increase in costs, greater hospitalization and above all the cost of the antifungals used, which together with the oxygen that is consumed to a large extent in this context, are the most expensive antibiotics. It should be noted that the arrival of covid through the pandemic is also a risk factor for acquiring this fungus, considering that our patient population is more at risk of acquiring the virus, even vaccinated, as well as that it evades the immune system. It would also be possible to make the filter effective on this virus. Prospectively, reports will be made about the evolution of its environmental sterilizing power (fungi, viruses and bacteria).

The only way to prevent the arrival of these spores, viruses and bacteria is with filtration into the ambient air of the same By virtue of the fact that it was already possible to have all the rooms with filters, a new reevaluation of respiratory infections by inhalation was made and it was found that they were not demonstrated since the arrival of all the filters in the rooms on the 11th floor, 10 in total in Room 3 of and 1 in Room 5 bed 5, Inhaled hospital-acquired infections linked to the acquisition in the Immunosuppressed Room. Unlike what happened in the previous months and in the previous years where there was a significant increase in hospital-acquired respiratory infections, especially pulmonary aspergillosis. From the placement of all the filters, in July to December (2022), in the Immunosuppressed rooms, their effectiveness was verified. Over July to December there was an estimated average of 40 discharges. The good evolution of the patients shows that positive results were obtained with the use of filters. The percentage of hospital-acquired infections (pneumonias) before the placement of ECOVIOX filters was 40% (to be confirmed with epidemiological data and at present only 2 patients presented lung infection due to hematogenous dissemination, which is not consistent with the inhalation mechanism.

### Example 5

Third report where the 12-month scientific study culminates. Begins in December 2022 until March 2023.

This report highlights the number of patients who passed through these areas in a period of two and a half months, an average of 37 patients. In one year, an average of 180 patients inhabits these areas. The infection rate was reduced by 100%, taking the infection cases from the years prior to the installation of the technology.

ROOM 3 REPORT OF IMMUNOSUPPRESSED PATIENTS (1S), FOURTH MEDICAL CLINICAL CHAIR. PROF. DR. SOFIA ISABEL FERNANDEZ

EVALUATION OF THE ecoVIOX AIR AND SURFACE DISINFECTION AND STERILIZATION SYSTEM.

Population to evaluate: Neutropenic patients, with alteration in quantity or quality of their neutrophils, secondary to chemotherapy or underlying disease (leukemia). Introduction: The patients are those that have altered any of their mechanisms of defense, either linked to your natural or adaptive immunity. The neutropenia is the decrease in neutrophil function (natural immunity) by disturbances in their quantity or quality. Regarding the amount according to IDSA 2011, it is required a decrease below 500/mm3 or in quality, which is the presence of blasts, in connection with leukemia. Although gram-negative sepsis, especially due to pseudomonas, increases the morbidity and mortality of these patients, there are other infections that position them in a condition of high vulnerability and poor prognosis. The historical experience in terms of our epidemiological data is the increase of probable invasive aspergillosis lung infection (it is enough even without the certainty that lung biopsy gives), to talk about said infection, the image plus the blood/lactomannan/BAL curve, and history of profound neutropenia and prolonged, are adequate for this diagnosis.

The arrival of the spores of said fungus to the lung of the patient with these conditions is an unavoidable situation in our environment due to environmental characteristics, devoid until March of filters to sterilize the air of these germs. Characteristically the neutrophil of the immunocompetent host has a enzymatic system that, when in contact with said fungus, manages to phagocytize it and eliminate it, a situation that cannot be implemented in the immunosuppressed patient due to neutrophil dysfunction, which lacks said enzymatic system, in quantity (absolute neutropenia) or quality (presence of blasts), where an immature system makes such debugging impossible.

Pulmonary aspergillosis increases the morbidity and mortality of patients with IS. In addition to being a pathology in itself serious for this population, it slows the chemotherapy treatment, that is, it worsens the prognosis of the underlying disease, as it also leads to superinfections and requiring OM transplantation, it is a factor that makes such a procedure difficult in the short and long term. As mentioned, if there are no adequate environmental filters, said infection will increasing, as evidenced by our statistics. As of March, three filters were placed in their respective rooms, assigned for admission of neutropenic patients. From that month up to and including June, they were not found in the rooms with filters. Pulmonary infections through upper airway access. A single patient developed a pulmonary nodule of probable arrival from infection of skin and soft parts (pathology with which he had been admitted). THERE WAS NO EVIDENCE OF PULMONARY ASPERGILLOSISN THIS GROUP OF PATIENTS FROM THE ACTION OF THE SEFILTERS, NOR OF OTHERS PULMONARY INFECTIONS.

In this same period, the presence of patients with aspergillosis had been documented lung, among other HI (hospital-acquired) infections in patients with 1S (before their placement).

As mentioned, not only does its presence have to do with morbidity and mortality, but also to the increase in costs, greater hospitalization and above all the cost of antifungals used, which together with the oxygen that is largely consumed in this context, they are the most costly antibiotics. It should be noted that the arrival of the covid through the pandemic is also a factor of risk to acquire said fungus, taking into account that our population of patients are at greater risk of acquiring the virus, even when vaccinated, as well as that itself evade the immune system. It would also fit the effectiveness of the filter on said virus. Reports will be made prospectively on the evolution of its sterilizing power. Environment (fungi, viruses, and bacteria). The only way to prevent the arrival of these spores, viruses and bacteria is with filtration. in the ambient air of the same.

By it was already possible to have all the rooms with filters, a new reassessment of respiratory inhalation infections and it was found that no demonstrated since the arrival of all the filters in the rooms on the 11th floor, 10th in total in Ward 3 and 1 in Ward 5 bed 5, nosocomial inhalation infections linked to acquisition in the Immunosuppressed Room. Unlike what occurred in the previous months and in the previous years where there was evidence of a significant increase in hospital-acquired respiratory infections, especially Pulmonary aspergillosis. From the placement of all the filters, in July to December (2022), in the Immunosuppressed rooms, their efficacy was verified. During July to December were an estimated average of 40 discharges. The good evolution of patients demonstrates that positive results were obtained with the use of the filters. The percentage of hospital-acquired infections (pneumonia) before the placement of the ECOVIOX devices was 40% (to be confirmed with epidemiological data) and in the currently, only 2 patients presented pulmonary infection by hematogenous dissemination, which is not consistent with the inhalation mechanism. During the months of January to March 2023, favorable results continued to be obtained with the use of ECOVIOX devices. Among 37 patients hospitalized in Room 3 during the mentioned period, no respiratory infections were recorded in any of them; to exception of a patient who was discharged for a study and who readmitted and presented with a probable lung infection. During this period the percentage of effectiveness was 100% since no cases were registered.

1. A device for medical room disinfection, comprising: an external housing; an upper intake for contaminated air; a lower outlet for purified air; a blower; at least one filter; at least one UV lamp; an ozonizer; and at least one positive ionizer.

2. The device of claim 1 wherein said filter comprises Honeycomb Active Carbon Filters Granule and a HEPA filter minimum width of 5 cm and a double silk pre-filter.

3. The device of claim 1 wherein said ozoniser comprising an UVC Lamp O₃.

4. The device of claim 1 comprising two lamps, a germicidal UVC lamp of 254 nm of at least 80 W, and another UVC O₃ lamp, generator of ozone gas.

5. The device of claim 1 wherein said ionizer comprises one generator of positive ions and a second generator of exclusively negative ions.

6. The device of claim 1 wherein said upper intake for contaminated air is located in the upper section of said housing and said lower outlet for purified air is situated in the lower section of said housing, wherein said lower outlet comprises two air exit vents driven by respective centrifugal turbines that suck in air, one exclusively with negative ions and the other with positive ions.

7. The device of claim 1 wherein said positive ionizer generates both negative ions and positive ions at a ratio ranging from 2/3 to 3/2 as negative ions/positive ions.

8. The device of claim 1 wherein it operates at a height of at least two meters, aspiring the air through its upper part and driving the purified air downwards.

9. The device of claim 1 wherein said activated carbon filter comprises granular, fiber or powdered activated carbon.

10. The device of claim 1 wherein said HEPA filters grades are selected from the group comprised by H11, H13 and H14.

11. The device of claim 1 wherein said ionizer is a plasma ionizer.

12. The device of claim 1 wherein said housing with mirrored inner surfaces comprises polished metal at least on its inner surface.

13. The device of claim 1 wherein said housing with mirrored inner surfaces comprises polished stainless steel at least on its inner surface.

14. The device of claim 1 wherein said UV lamps are selected from the group comprised by: LED technology lamps and mercury vapor lamps.

15. The device of claim 1 wherein said blower comprises two centrifugal turbines commanded by a single electric motor located between said turbines that operates at 2800 rpm and generates an air flow of from 400 to 2000 m3/h.

16. The device of claim 15 wherein said two ionizers, one positive ionizer and the other negative ionizer are placed in the suction of each centrifugal blower, keeping two air streams with different charge.

17. A device for medical rooms disinfection, comprising:
an external housing;
an upper intake for contaminated air;
a lower outlet for purified air with two vents;
an electronic controller with a timer;
a blower with two centrifugal turbines;
a HEPA filter and an activated carbon filter;
at least one UV lamp;
an ozonizer;
a positive ionizer located in the suction of one of the centrifugal turbines and a negative ionizer located in the suction of the other centrifugal turbine.

18. The device of claim 17 wherein said electronic controller with a timer controls the intermittent operation of the ozonizer so as not to exceed the maximum ozone concentration allowed in a working day.

19. A method for disinfecting air in medical rooms using the device of claim 1 wherein said device is preferably installed at a distance of at least 2 m and less than m from the floor and comprises the following steps:
a. drawing in the air from the medical room to be purified through the upper intake;
b. forcing the air drawn in step “a” to pass through the honeycomb type activated carbon filter, then through the pleated HEPA filter h14 of at least 5 cm in thickness, and through a UV lamp of at least 80 W;
c. splitting the air stream into two, where one of the two streams passes through a positive ionizer and the other stream passes through a negative ionizer;
d. turning on the ozonizer for no more than 20 min. every 4 hours;
e. expelling the purified, ionized, and disinfected air through a lower outlet.

20. The method of claim 18 wherein said UV lamp is UVC and irradiates said HEPA filter.

21-22. (canceled)
